(19) United States
(12) Patent Application Publication
(10) Pub. No.: US 2006/0224073 A1
(43) Pub. Date: Oct. 5, 2006

(54) INTEGRATED PHYSIOLOGICAL SIGNAL ASSESSING DEVICE

(75) Inventors: Kang-Ping Lin, Jhongli City (TW); Hao Yu Jan, Yonghe City (TW); Bor-Iuan Jan, Pingtung City (TW)

Correspondence Address: NIKOLAI & MERSEREAU, P.A., 900 SECOND AVENUE SOUTH, SUITE 820, MINNEAPOLIS, MN, 55402, US

(73) Assignee: DAILYCARE BIOMEDICAL INC., Chung-Li City (TW)

(21) Appl. No.: 10/907,355

(22) Filed: Mar. 30, 2005

(51) Int. Cl. A61B 5/0402

(52) U.S. Cl. 600/513

(57) ABSTRACT

An integrated physiological signal assessing device integrates electrical signal detecting technology and optical detecting technology to design a sensing interface module for simultaneously measuring ECG signals and PPG signals, and utilizes multiple algorithm processing methods to obtain ECG parameters, such as rhythm of a heart, ST segment, and QRS interval, as well as vascular parameters, such as vascular stiffness index (SI), vascular reflection index (RI), pulse wave velocity (PWV), and pulse oxygen saturation (SpO₂), so as to simplify the conventional vascular functions measuring devices, easily understand physiological conditions of a subject, and helpfully apply on diagnosis and prevention of cardiovascular diseases.

### BACKGROUND OF THE INVENTION

1. Field of the Invention

The present invention relates to a physiological signal assessing device, particular to an integrated physiological signal assessing device for simultaneously measuring electrocardiographic signals (ECG signals) and photoplethysmographic signals (PPG signals). The device utilizes a sensing interface, cooperating with subsequent signals analysis and processing, to conveniently obtain many useful physiological parameters, such as ECG signals, pressure pulse, pulse oxygen saturation etc.

2. Description of Related Art

Nowadays, people enjoy more delicate and delicious food, but lack sufficient exercise habits so the factors of cardiovascular diseases have increased recently. Besides, although the biotech and medical advances have prolonged people's lives, the functions of the cardiovascular system of people will still be weaker with their increasing age. Thus, in recent years relevant cardiovascular diseases have become great threats to the health of people.

In terms of medical equipment, the diagnosis of a heart is mainly based on use of a cardiograph. The cardiograph is able to record the electrical transmittal process of cardiac muscles when the cardiac muscles take rhythmic systole, and express the results as an electrocardiogram, by which a doctor can judge if the functions of the heart are normal or not. On the other hand, an oximeter is the most general equipment used to evaluate the pulse oxygen saturation (SPO₂) which can estimate the function of blood circulation and the oxygen-providing situation in blood, and is also an important index of the amount of oxygen for transmittal and metabolism in brain tissues. In addition, there are two indexes, that is, large artery stiffness index (SI) and vascular reflection index (RI) separately, obtained from the analysis of pressure pulse to estimate the aging degrees of blood vessels and the resilient functions of blood vessels. Matching up the pressure pulse with the ECG signals can obtain pulse wave velocity (PWV) to further know the relationship between the blood vessels and the blood flow.

Clinically, a doctor usually uses a cardiograph with at least three electrodes to record multiple leads measurement to obtain more detailed electrocardiographic signal transmittal data. However, from the ECG signal measuring principle, using two electrodes is able to obtain single vector ECG signals.

As to pressure pulse, with reference to FIG. 1 taking a finger for example to illustrate the relationship between the pulse and the blood vessels, the pulse waveform of the finger can be divided into two parts. The first part is caused from that the pulse, along the aorta, is directly transmitted to the finger while the second part is caused from that the pulse is transmitted to the lower body and then reflected back along the aorta and subclavian artery to the finger. The time delay between the first peak and the second peak of the pulse is mainly determined by transmit time of the pulse transmitted back and forth along the subclavian artery. The transmit time is directly proportional to a subject height and related to the resilience of blood vessels. The more resilient the blood vessels are, the better the ability to absorb the pulse the blood vessels have, such that the transmittal time of the reflected pulse is longer and the pulse wave velocity is slower. Therefore, from the pressure pulse, the resilience conditions of the blood vessels can be known.

Today there are two major ways of detecting the pulse waveform: one is by pressure; the other is by optical means. The pressure method is similar to the blood pressure measurement, i.e. using a cuff to wrap around a tested portion of the subject and after pumping air into the cuff to compress the tested portion, then detecting the variations of the pulse by means of a pressure sensor, as disclosed in U.S. Pat. Nos. 6,802,814; 6,758,819; and 6,758,820 to Colin Medical Tech. Corp. The corporation's product of VP-1000/2000 also utilizes the pressure method to measure the ratio of an ankle's blood pressure to an upper arm's blood pressure, and the pulse wave velocity (PWV).

The optical method mainly makes use of the characteristics of light, such as reflection, absorption, transmission, etc. Taking infrared rays for example, oxygenated blood can absorb more amounts of infrared rays than deoxygenated blood. When the heart is systolic, the amounts of oxygenated blood, with faster movement velocity, in an artery are increased such that the blood absorbs more infrared rays. The situation is to the contrary when the heart is diastolic. This type of optical method of using light to irradiate a subject's body, receiving and recording the optical signals from the blood vessels with time and tissue's variations is called photoplethysmography (PPG). The PPG is not only related to oxygen concentrations in the blood but also responds to the variations of the pulse, and further is used to calculate SI, RI, PWV, and SPO₂. U.S. Publication No. 2004/0015091, for example, has disclosed utilizing an optical probe to obtain PPG.

In the market, there is PPG-related equipment to detect the pressure pulse waveform so as to calculate relevant parameters about vessel functions. For example, Micro Medical (U.S.) published an instrument “Pulse Trace PCA (PT2000)” utilizing an optical probe to detect pressure pulse and further providing SI value; and an instrument “Pulse Trace PWV (PT400) simultaneously using three electrodes and a Doppler probe to obtain the subject's electrocardiogram and pressure pulse so as to calculate PWV.

These above-mentioned parameters, such as, SI, RI, PWV, and SPO₂ etc., are very helpful to estimate the subject's cardiovascular system conditions and are often used in clinical diagnosis. Nevertheless, based on different measurement principles, conventional instruments have to use different sensor elements to separately obtain ECG signals, pulse oxygen saturation, and pressure pulses, so if all the cardiovascular indexes and parameters need to be obtained, several various measuring instruments have to be used, which is time-consuming and inconvenient.

In order to resolve the problems existing in the prior art to effectively and simply obtain the cardiovascular parameters, the present invention integrates the optical measurement principle and the electrical measurement principle to design an integrated measuring instrument with a sensing interface combining sensing electrodes with an optical probe, such that the instrument is able to simultaneously obtain ECG signals and PPG signals, further acquire oxygen concentration in blood, and through algorithm processing and analysis of the signals gain all cardiovascular parameters.

### SUMMARY OF THE INVENTION

It is an objective of the present invention to provide an integrated physiological signal assessing device with an integrated sensing interface for optical and electrical measurement to simultaneously get ECG signals and PPG signals to obtain an electrocardiogram, pressure pulse waveforms, and pulse oxygen saturation.

It is another objective of the present invention to provide a design that is able to evaluate parameters about cardiovascular functions by analyzing and calculating the ECG signals and the PPG signals.

It is yet another objective of the present invention to provide a sensing interface which integrates the ECG signal sensing technology and the optical sensing principle to get multiple physiological signals, such as ECG signals, pressure pulse waveforms, and pulse oxygen saturation.

It is yet another objective of the present invention to provide a device with a sensing interface for measuring and analyzing cardiovascular parameters. The device not only gets ECG signals but also simultaneously records pressure pulses and pulse oxygen saturation, and further, through calculation and analysis, obtains cardiovascular parameters, such as heart rate, ST segment, QRS interval, pulse oxygen saturation, stiffness index, reflection index, pulse wave velocity . . . etc.

To attain such objectives, the present invention is based on double electrode ECG signal measuring technology to combine inventively with optically physiological signal measurement to design a sensing interface to simultaneously get ECG signals and PPG signals to serve as the basis for calculating and analyzing cardiovascular parameters, thereby simplifying the conventional instrument or equipment and facilitating a user to monitor one self's cardiovascular conditions.

Other and further features, advantages and benefits of the invention will become apparent in the following description taken in conjunction with the following drawings. It is to be understood that the foregoing general description and following detailed description are exemplary and explanatory but are not to be restrictive of the invention. The accompanying drawings are incorporated in and constitute a part of this application and, together with the description, serve to explain the principles of the invention in general terms. Like numerals refer to like parts throughout the disclosure.

### BRIEF DESCRIPTION OF THE DRAWINGS

The objects, spirits and advantages of the preferred embodiments of the present invention will be readily understood by the accompanying drawings and detailed descriptions, wherein:

FIG. 1 is a schematic view showing the relationship between pressure pulse waveform and pulse transmit path;

FIG. 2 is a schematic view of pressure pulse waveform;

FIG. 3 is a schematic view showing the relationship between pressure pulse waveform and vascular stiffness degree;

FIG. 4 is a schematic view showing the relationship between pressure pulse waveforms and ECG signals;

FIG. 5 is a block diagram of the device according to the present invention;

FIG. 6A is a schematic view of a sensing electrode structure according to the present invention;

FIG. 6B is a measuring schematic view of the sensing electrode;

FIG. 7 is a schematic view of another embodiment of a sensing electrode structure;

FIG. 8 is a more detailed block diagram of the present invention; and

FIG. 9 is a flow chart of algorithm of parameters operated in the present invention.

### DETAILED DESCRIPTION OF THE PREFFERED EMBODIMENT

An integrated physiological signal assessing device constructed in accordance with the present invention mainly utilizes an electric signal sensing principle and an optical signal sensing principle, and designs a sensing interface combining different sensing elements to get ECG signals, pressure pulses, and pulse oxygen saturation index.

Referring to the optical signal sensing principle of pressure pulse, Beer-Lambert's law has to be mentioned. The law indicates that if light with a certain wavelength is absorbed by dissolved material in solvent, the amount of transmission light (I) is decreased exponentially with the product of dissolved material concentration and transmission distance in the solvent, the formula as follows:
I=I₀e^{-εcd} (1)

wherein ε is absorption coefficient.

Equally, when light passes through a subject's tissue, the skin, muscles, bones, and blood will absorb certain amount of light. The amount of light absorbed by the skin, muscles, and bones is fixed, but is varied with the oxygen concentration in the blood. Because of the effect of the circulatory system, that is, deoxygenated blood exchanging air in the lungs to become oxygenated blood and being transferred over the whole body, the PPG technology is able to record the variations of oxygen concentration in the blood. Furthermore, since the oxygen concentration in the blood is varied with pressure pulse, thereby pressure pulse waveforms can be inferred from the variations of the oxygen concentration.

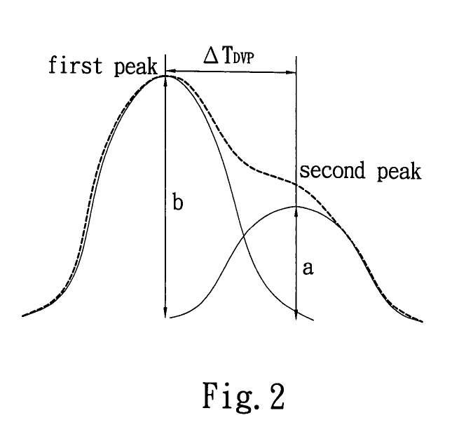

Taking a pressure pulse waveform of PPG of a subject's finger for example, as shown in FIG. 2, time delay between the first peak and the second peak depends on the transmit time of the pulse transmitting along a subclavian artery to a lower body of the subject and reflecting back to the subclavian artery. Assuming the pulse transmit distance is directly proportional to a subject's height, and the pulse transmit time from an aorta to large arteries is related to the resilience of the blood vessel, therefore, the large artery stiffness can be estimated in following formula:

$${SI}_{DVP} = \frac{Subject height}{Δ {T}_{DVP}} in ms \qquad ( 2 )$$

SI: vascular stiffness index;
ΔT_{DVP}: delayed time between two peaks in a pulse waveform;
DVP: digital volume pulse.

In addition, a difference between heights of the two peaks is used to estimate the reflection intensity of reflected blood transmitted back in the artery, namely RI calculated as the following formula:

$${RI}_{DVP} = \frac{a}{b} ⨯ 100 \% \qquad ( 3 )$$

a: height of the second peak;
b: height of the first peak.

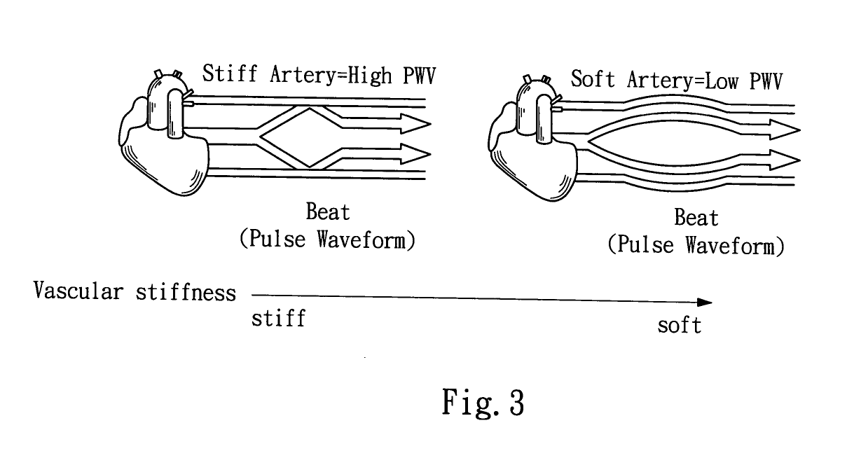

The values of Si and RI respectively in formula (2) and (3) can be gained by the pressure pulse waveform. However, if one would like to gain PWV, the ECG signals are necessary to be cooperated with. Since PWV is used to estimate the velocity of the pulse which is generated by the heart, passed through blood vessels, and transmitted to hands and legs, the greater the PWV is, the stiffer the blood vessel is. Thus, PWV is significantly related to SI. With reference to FIG. 3, if the blood vessel becomes stiff like ceramics, the pulse is not easy to be absorbed by the blood vessel such that the velocity of the pulse is faster. On the other hand, if the blood vessel is resilient like rubber, the pulse can be absorbed by the wall of the vessels such that the velocity of the pulse is slower. Research reports point out that PWV is greatly related to cardiovascular diseases, indicating that the greater value of PWV results in the larger probability of a subject suffering of coronary artery disease.

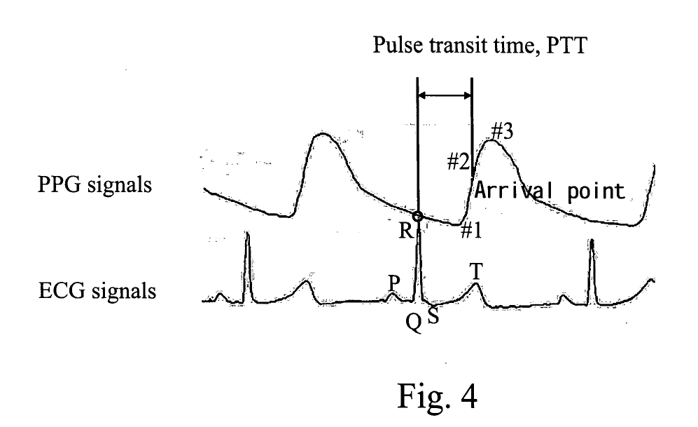

Calculation for PWV is as follows, with reference to FIG. 4:

$$PWV = \frac{D}{PTT} \qquad ( 4 )$$

D: pulse transmit distance;
PTT: pulse transmit time.

As shown in FIG. 4, to calculate PWV, it is necessary to combine with ECG signals to know where blood begins to be transferred out of the heart. In ECG signals, the “R” wave is easier to be detected, so the R wave signal is generally used as a mark of time starting point.

Although using light with one wavelength is able to get a PPG to know the variation of pressure pulse, it is necessary to use light sources with at least two different wavelengths to get two PPGs to obtain pulse oxygen saturation (SPO₂), which is an important parameter for circulatory functions. When a person takes in breath, oxygen outside of the subject's body enters into the trachea, whereafter it is transferred to the pulmonary alveolus to be exchanged into blood, and then is transferred around the whole body to provide for the tissues. SPO₂ is mainly used to estimate the concentration of oxygenated hemoglobin. Since blood in the body consists of two types: oxygenated blood and deoxygenated blood, it is necessary to use two types of light sources with different wavelengths to separately measure the concentration of oxygenated hemoglobin and the concentration of deoxygenated hemoglobin, and then evaluate the percentage of the oxygenated hemoglobin as the following formula:

$${SpO}_{2} = \frac{[ {HbO}_{2} ]}{[ {HbO}_{2} ] + [ Hb ]} ⨯ 100 \% \qquad ( 5 )$$

HbO₂: concentration of oxygenated hemoglobin; and
Hb: concentration of deoxygenated hemoglobin.

In order to calculate the concentration of oxygenated hemoglobin and deoxygenated hemoglobin, formula (1) has to be used. For convenient computing, there is a variable OD defined herein, so formula (1) is rewritten as follows:

$$OD = ln ( \frac{{I}_{0}}{{I}_{1}} ) = ɛ c d \qquad ( 6 )$$

Red light and infrared light, especially respectively about 660 nm and 940 nm, are commonly used to calculate SPO₂, so the formula (6) can be expressed as:
OD(660)=ε_{HbO}_{₂}(660)c_{HbO}_{₂}d+ε_{Hb}(660)c_{Hb}d
OD(940)=ε_{HbO}_{₂}(940)c_{HbO}_{₂}d+ε_{Hb}(940)c_{Hb}d (7)

Thus,

$${SpO}_{2} = \frac{{Rs}_{Hb} ( 940 ) - {ɛ}_{Hb} ( 660 )}{R {ɛ}_{Hb} ( 940 ) - {ɛ}_{Hb} ( 660 ) + {ɛ}_{{HbO}_{2}} ( 940 ) - {ɛ}_{{HbO}_{2}} ( 660 )} \\ wherein R = \frac{OD ( 660 )}{OD ( 940 )} \qquad ( 8 )$$

Formulae (2) to (8) are respectively for computing SI, RI, PWV, and SPO₂, and with prior art, these different parameters have to be measured by different devices with various designs of sensing elements. An integrated physiological signal assessing device of the present invention develops an interface with a design of integrating a double electrode sensing principle of ECG signal and an optical sensing probe, to get ECG signals, pressure pulse, and SPO₂, thereby simplifying medical equipment and being able to apply in family.

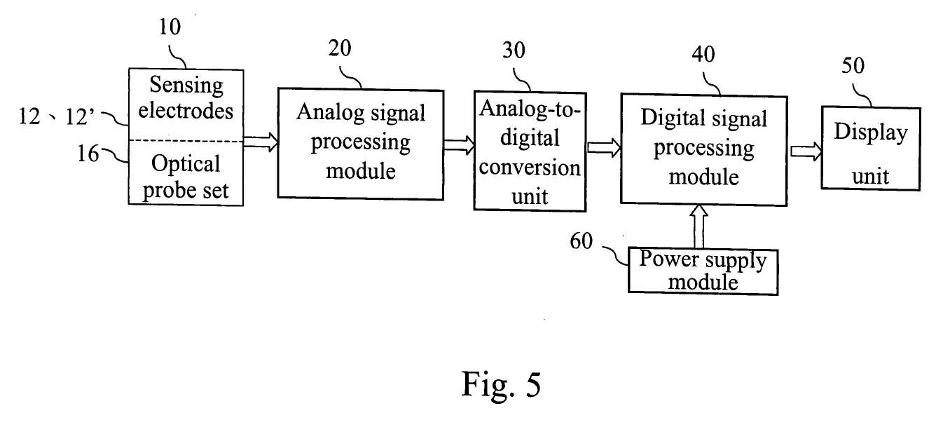

FIG. 5 is a block diagram of the integrated physiological signal assessing device according to the present invention. The device comprises a sensing interface module (10), an analog signal processing module (20), an analog-to-digital conversion unit (30), digital signal processing module (40), and a display unit (50). The sensing interface module (10) includes two sensing electrodes (12, 12′) and an optical probe set (16). The sensing electrodes (12, 12′) are used to measure ECG signals, while the optical probe set (16) combined with at least one of the sensing electrodes (12, 12′) is used to measure PPG signals. The detailed design of the sensing interface module (10) will be described hereafter.

The analog signal processing module (20) is electrically connected to the sensing interface module (10) and used to process the ECG signals and PPG signals that are in analog forms and measured from the subject, to amplify and filter these signals. The analog-to-digital conversion unit (30) is used to convert the analog ECG signals and the analog PPG signals, all of which have been processed by the analog signal processing module (20), to digital signals respectively. These digital signals then are processed by the following digital signal processing module (40) carrying out various kinds of algorithm so as to obtain ECG parameters, such as heart rate, ST segment, QRS interval, etc., and vascular parameters, such as SI, RI, PWV, SPO₂, etc. This information of such parameters can be transmitted to show on a display unit (50), such as liquid crystal display, LED, etc. The device can further have a power system (60), such as electric cell set or external power source like domestic socket, to supply power for all electricity-consuming elements as described above.

In the present invention, the sensing interface module (10) is a very inventive design to simultaneously get two different types of signals: ECG signal and PPG signal. Since there two optical methods, that is, reflection way and transmission way, to detect PPG signals, the structure of the sensing interface module (10) has different embodied manners. In addition, because at least two electrodes are possible to record an ECG signal but only at least one optical probe is able to record pressure pulse and SPO₂, the device can comprise either one optical probe set (16) disposed on one of the sensing electrodes (12, 12′) or more than one optical probe disposed on both of the sensing electrodes (12, 12′). In the following embodiment, there is only one optical probe set (16) disposed on one of the sensing electrodes (12, 12′).

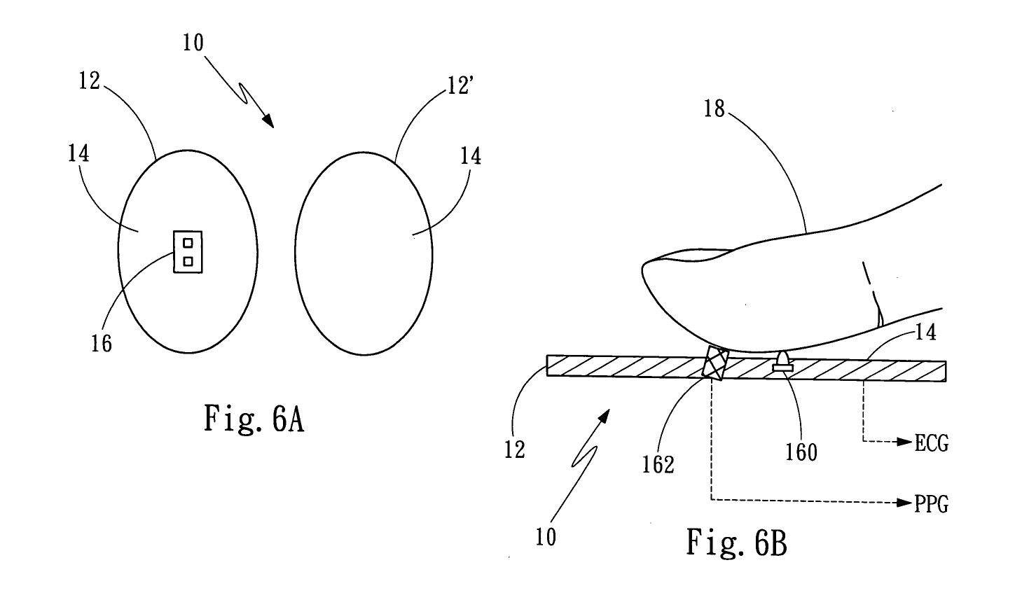

In an optical reflection manner, as shown in FIG. 6A, the sensing interface module (10) comprises two sensing electrodes (12, 12′) and an optical probe set (16) disposed on one of the sensing electrodes (12, 12′). Each of the sensing electrodes (12, 12′) has a contact surface (14) to contact the subject's body surface to record the variations of current when the subject's heart muscles rhythmically contract, so as to get ECG signals. The optical probe set (16) includes at least one light generator (160) and at least one light receiver (162). The light generator (160) is used to emit rays of light to the subject's body surface, and the light receiver (162) is used to receive the reflected light from the subject's tissues to form PPG signals.

In this manner, the sensing interface module (10) can be designed in a plane style to be fixedly adhered on the subject's body surface, or easily be contacted by the subject. With reference to FIG. 6B, taking the subject's finger as the tested portion for example, when the finger (18) is set on the sensing electrodes (12, 12′) (sensing electrode 12′ not shown in FIG. 6B), the sensing electrodes (12, 12′) can receive the ECG signals transmitted from the heat and record thereof. For twelve-lead ECG measurement, the signals from the fingers of both hands belong to the first lead ECG signals. The light generator (160) and the light receiver (162) are both combined with one of the sensing electrodes (12, 12′), so if the pressure pulses need to be known, the light generator just (160) needs to emit rays of light, for example red rays of light or infrared rays of light, to the finger, and receive and record the amounts of the reflected light to get PPG signals based on variations of concentration of oxygen in blood. By following modules to process these obtained PPG signals, SI and RI can be calculated. Furthermore, by measuring ECG signals and PPG signals at the same time and combining them together, PWV also can be computed. To estimate the concentration of oxygen in blood, the light generator (160) has to emit two types of light sources with different wavelengths, generally the red rays of light and the infrared rays of light, and then the light receiver (162) receives, in a clock-switching manner, the two types of reflected light sources with different wavelengths respectively with respect to oxygenated blood and deoxygenated blood, so as to provide enough information for the following modules to calculate SPO₂.

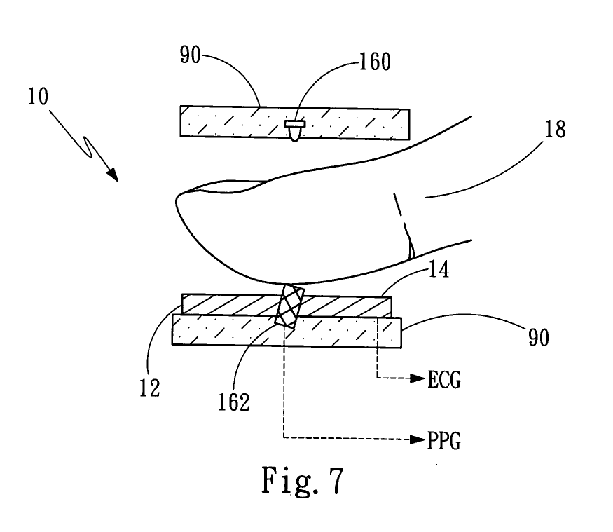

With reference to FIG. 7A which is another embodiment showing a cross-sectional schematic view of transmission type of sensing interface module (10), the sensing interface module (10) further comprises a fixture (90), such as a ring, a finger sheath . . . etc., having two corresponding and opposite inner faces. At least one of the sensing electrodes (12, 12′) is enveloped therein and attached on the one of the inner faces of the fixture (90) with the contact surface (14) facing the subject's body surface. The light generator (160) and the light receiver (162) are also enveloped in the fixture (90) and respectively disposed on the opposite inner faces. In this embodiment, the optical probe set (16) and the sensing electrode (12) are enveloped in the same fixture (90). The light receiver (162) is disposed on the sensing electrode (12) on the bottom inner face of the fixture (90), while the light generator (160) is mounted on the opposite inner face of the fixture (90). Of course, the light generator (160) and the light receiver (162) can exchange with each other. After the rays of light emitted from the light generator (160) are transmitted to the subject and pass through the subject's tissues, the light receiver (162) can receive the transmission light. When using this type of measuring way, the subject sets one hand's finger on the contact surface (14) of the sensing electrode (12′) (not shown in FIG. 7B), and sets a finger of the other hand in the fixture (90) to touch the contact surface (14) of the sensing electrode (12). By this way, the subject's ECG signals can be obtained by the sensing electrodes (12, 12′), and the PPG signals can also be obtained by using single wavelength light, or two wavelength lights and clock-switching manner, as described above, to provide for the following modules to calculate RI, SI, PWV, and SPO₂.

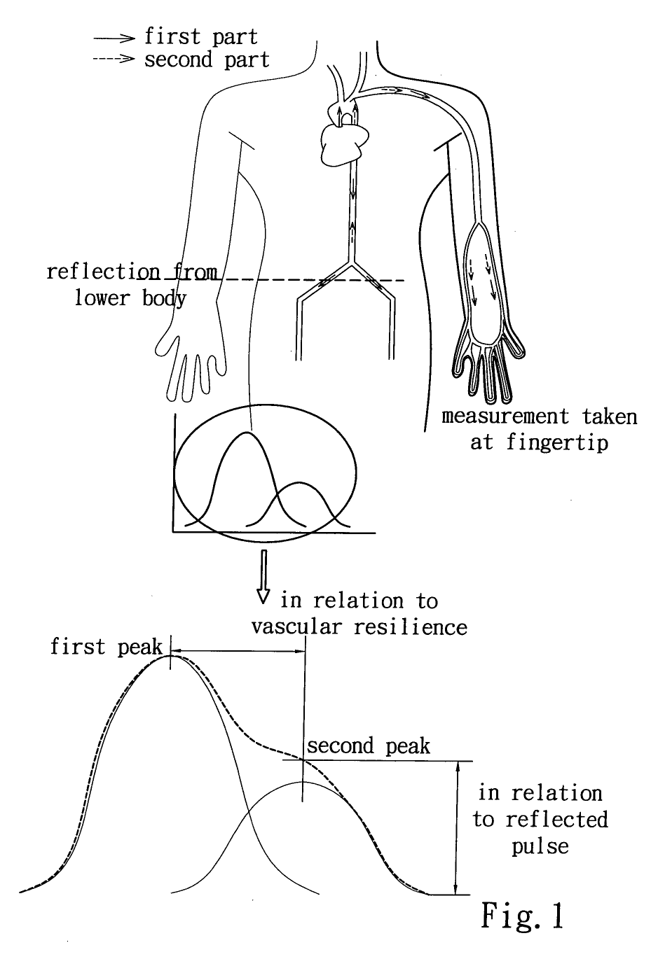

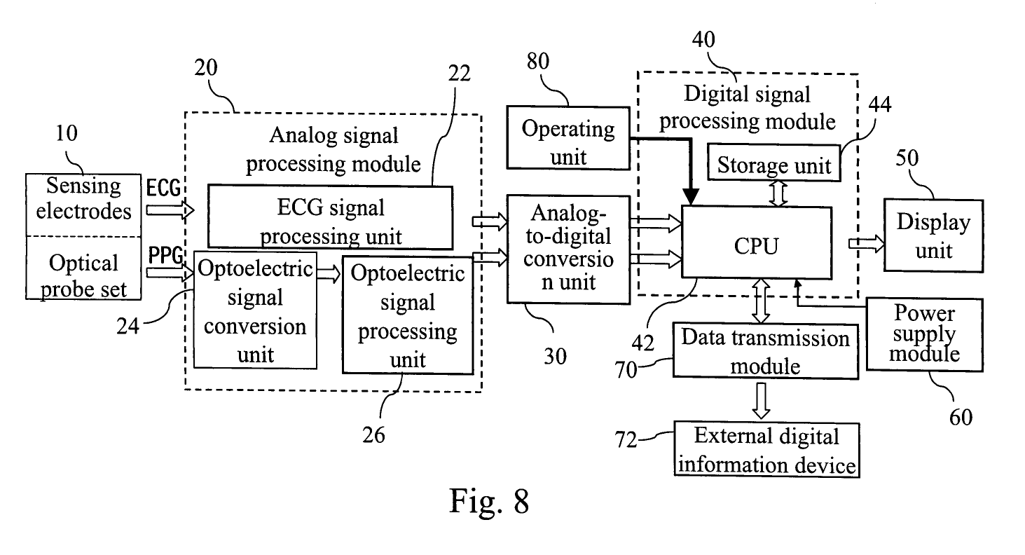

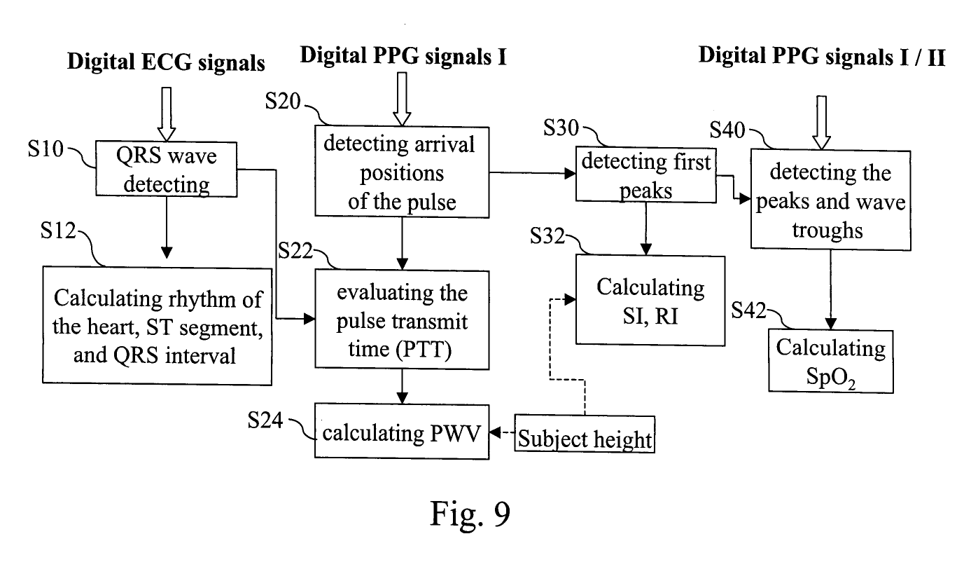

With reference to FIG. 8 which is a more detailed block diagram of the present invention, when the subject uses the present invention, the sensing electrodes (12, 12′) of the sensing interface module (10) can get the subject's ECG signals and the optical probe set (16) can get the subject's PPG signals at the same time. After that, the analog signal processing module (20) connected electrically to the sensing interface (10) deals with the ECG signals and the PPG signals separately. In the analog signal processing module (20), there are an ECG signal processing unit (22) to amplify and filter the ECG signals, an optoelectronic signal conversion unit (24) to convert the optical signals of PPG to electrical signals, and an optoelectronic signal processing unit (26) to amplify and filter the converted PPG signals. The ECG signals and the PPG signals are then electrically transmitted to the analog-to-digital conversion unit to be converted to digital signals. These ECG and PPG digital signals are then transmitted to the digital signal processing module (40) to proceed to calculate various physiological parameters. The digital signal processing module (40) comprises of a CPU (42) for further processing the ECG signals and PPG signals to obtain ECG parameters, such as heart rate, ST segment, and QRS interval, and obtain PPG parameters, such as SI, RI, PWV, and SPO₂, and so on. The detailed algorithm process is illustrated in FIG. 9 and FIGS. 1 to 4 are referred to for clear understanding.

When the digital ECG signals are transmitted to the CPU (42), the ECG signals processing procedure enters the first step (SI 0), i.e., detecting the QRS wave from the ECG signals. Normally, the ECG signals are composed of a P wave, a QRS wave, and a T wave. However, the QRS wave has stronger intensity to be easily detected resulting from being caused by depolarized current before ventricular systole. In addition, the R wave is the basis for rhythm of the heart, so the first step is to detect the position of the QRS wave. Then, by means of computing voltage of the whole ECG signals and detecting QRS signals, ECG parameters are calculated, such as heart rate, ST segment, QRS section, and so on, as in step (S12).

For single light source PPG signals, when the PPG signals are transmitted to the CPU (42), the PPG signals processing procedure enters the following steps. The first step is detecting arrival positions of the pulse, step (S20). There is no uniform standard to determine the arrival position presently, but generally the turning point to rise, the point of the greatest rising slope, or the peak of the pulse as the arrival position of the pulse (as respectively indicated at #1, #2, and #3 in FIG. 4) is taken. The embodiment takes the point with greatest rising slope as the arrival position of the pulse for example herein. The next step is comparing the digital PPG signals with QRS waves of the ECG signals to evaluate the pulse transmit time (PTT) from the peak of the QRS wave to the arrival position of the pulse, step (S22). The subsequent step is using formula (4), as well as estimating the subject height and the tested portion of the subject, to obtain PWV, i.e., step (S24). Besides, the step (S20) is also followed by another step (S30) of detecting a first peak and a second peak of the PPG signal to compute the amplitudes of the first peak and the second peak, and an interval of time between the two amplitudes. The following step (S32) is executing algorithm for calculating formulae (2) and (3) to obtain SI and RI. The PWV, SI, and RI can be obtained by single light source of PPG signals. Moreover, if having two different light sources, for example the red light source and the infrared light source, the CPU (40) can further execute another step, following the step (S30), of determining the amounts of light absorption or reflection with respect to the different light sources (S40), that is, detecting the peaks and wave troughs of the different types of PPG signals. Then, next step is calculating formulae (5) to (8) to obtain SPO₂.

After the CPU (42) calculates the ECG parameters and vascular parameters, these parameters are transmitted to the display unit (50), such as an LCD, LED, etc., to show thereon visible information for the subject. The digital signal processing module (40) further comprises a storage unit (44) connected electrically to the CPU (42) to save the digital ECG signals, the digital PPG signals, the ECG parameters, and the vascular parameters. The digital signal processing module (40) is further connected to a data transmission module (70), such as USB interface, Bluetooth interface, infrared rays interface, modem, etc., to transmit such data including the signals and parameters saved in the storage unit (44) to an external digital information device (72), such as a personal computer, a PDA, a cell phone, database, etc. for providing subsequent diagnosis and analysis, and data management.

The integrated physiological signal assessing device further comprises an operating unit (80) electrically connected to the CPU (42) to have the subject be able to control the operation of the digital signal processing module. The operating unit (80) can be presented in any manner, such as buttons, knobs, touch panels, etc., to carry out the desired actions, such as performing measuring functions, adding/deleting/transmitting the data in the storage unit (44), inputting the subject's personal information, setting a date, etc. By means of the power supply module (60) to provide power for the whole measuring device, all the modules and units can successfully operate so as to achieve the effects of detecting and analyzing several different types of signals.

Although this invention has been disclosed and illustrated with reference to particular embodiments, the principles involved are susceptible for use in numerous other embodiments that will be apparent to persons skilled in the art. This invention is, therefore, to be limited only as indicated by the scope of the appended claims.

What is claimed is:

1. An integrated physiological signal assessing device comprising:
a sensing interface module comprising two sensing electrodes for obtaining a subject's ECG signals, and at least one optical probe set selectively combined with one of the sensing electrodes for simultaneously getting the subject's PPG signals when obtaining the ECG signals;
an analog signal processing module electrically connected to the sensing interface module for analogically processing the ECG signals and PPG signals detected by the sensing interface module;
an analog-to-digital conversion unit electrically connected to the analog signal processing module for converting the ECG signals and the PPG signals in analog forms to digital ECG signals and digital PPG signals;
a digital signal processing module electrically connected to the analog-to-digital conversion unit and having a CPU for executing algorithm separately with respect to the digital ECG signals and the digital PPG signals to obtain at least one ECG parameter and at least one vascular parameter;
a display unit electrically connected to the digital signal processing module to display the ECG parameter and the vascular parameter; and
a power supply module providing power for all elements described above.

2. The integrated physiological signal assessing device of claim 1, wherein each sensing electrode has a contact surface to contact the subject's body surface to obtain the ECG signals.

3. The integrated physiological signal assessing device of claim 1, wherein the optical probe set comprises at least one light generator for emitting light to the subject, and at least one light receiver for receiving light transmitted from the subject.

4. The integrated physiological signal assessing device of claim 3, wherein the light generator and the light receiver are disposed on the same sensing electrode.

5. The integrated physiological signal assessing device of claim 3, wherein when the light generator is disposed on the sensing electrode, the light receiver is located over the light generator, and when the light receiver is disposed on the sensing electrode, the light generator is located over the light receiver.

6. The integrated physiological signal assessing device of claim 3, wherein the light emitted from the light generator is red light or infrared light.

7. The integrated physiological signal assessing device of claim 1, wherein the ECG parameter is rhythm of a heart, ST segment, or QRS interval.

8. The integrated physiological signal assessing device of claim 1, wherein the vascular parameter is pulse oxygen saturation, vascular stiffness index, vascular reflection index, or pulse wave velocity.

9. The integrated physiological signal assessing device of claim 1, wherein the analog signal processing module comprises:
an ECG signal processing unit for amplifying and filtering the ECG signals detected from the subject;
an optoelectronic signal conversion unit for converting the optical PPG signals detected from the subject to electrical PPG signals; and
an optoelectronic signal processing unit for amplifying and filtering the electrical PPG signals.

10. The integrated physiological signal assessing device of claim 1, wherein the digital signal processing module further comprises a storage unit electrically connected to the CPU, to save the digital ECG signals, the digital PPG signals, the ECG parameters, and the vascular parameters.

11. The integrated physiological signal assessing device of claim 10, wherein the digital signal processing module further is connected to a data transmission module to transmit the signals and parameters saved in the storage unit to an external digital information device.

12. The integrated physiological signal assessing device of claim 11, wherein the data transmission module is a USB transmission interface, Bluetooth™ transmission interface, infrared rays transmission interface, or modem.

13. The integrated physiological signal assessing device of claim 11, wherein the external digital information device is a personal computer, a personal digital assistant, medical database, or a cell phone.

14. The integrated physiological signal assessing device of claim 1, wherein the CPU is further connected to an operating unit for having the subject control the actions of the digital signal processing module.

15. The integrated physiological signal assessing device of claim 1, wherein the power supply module is a cell set or external power source.

16. A sensing interface module for an integrated physiological signal assessing device, the module comprising:
two sensing electrodes having a respective one of contact surfaces to contact a subject's body surface for obtaining the subject's ECG signals;
at least one optical probe set combined with one of the sensing electrodes for simultaneously acquiring the subject's PPG signals when obtaining the ECG signals.

17. The sensing interface module of claim 16, wherein the optical probe set comprises at least one light generator for emitting light to the subject, and at least one light receiver for receiving light transmitted from the subject.

18. The sensing interface module of claim 17, wherein the light generator and the light receiver are disposed on the same sensing electrode.

19. The sensing interface module of claim 17, wherein when the light generator is disposed on the sensing electrode, the light receiver is located over the light generator, and when the light receiver is disposed on the sensing electrode, the light generator is located over the light receiver.

20. The sensing interface module of claim 17, wherein the light emitted from the light generator is red light or infrared light.
